(19) United States
(12) Patent Application Publication
(10) Pub. No.: US 2025/0194572 A1
(43) Pub. Date: Jun. 19, 2025

(54) AUTOMATIC BREEDING CONTROL SYSTEM FOR BLACK SOLDIER FLIES

(71) Applicants: MENG-KUN WU, CHIAYI COUNTY (TW); SHU-MIN CHEN, CHIAYI COUNTY (TW)

(72) Inventors: MENG-KUN WU, CHIAYI COUNTY (TW); SHU-MIN CHEN, CHIAYI COUNTY (TW)

(21) Appl. No.: 18/545,173

(22) Filed: Dec. 19, 2023

(51) Int. Cl. A01K 1/00

(52) U.S. Cl. CPC A01K 67/30

(57) ABSTRACT

An automatic breeding control system for black soldier flies includes a conveying unit, having a breeding space and two outlets; a lifting unit, being selectively moved closer to or away from the conveying unit in a height direction, the lifting unit having two outlet baffles and a plurality of rakes between the outlet baffles; at least one infrared temperature sensor, disposed on the lifting unit; a control unit, being in signal connection with the conveying unit, the lifting unit and the infrared temperature sensor. The rakes can stir black soldier fly larvae in the breeding space in a timely manner to prevent the black soldier fly larvae from dying because the temperature of the breeding space is too high.

### FIELD OF THE INVENTION

The present invention relates to an automatic breeding control system for black soldier flies, and more particularly to an automatic breeding control system which can stir the black soldier fly larvae in a breeding space in a timely manner so as to avoid the death of the black soldier fly larvae due to poor heat dissipation.

### BACKGROUND OF THE INVENTION

The black soldier fly is a detritivorous insect of the family Stratiomyidac. Detritivorous insects feed on rotten food, such as rotten fruits, animal feces or dead bodies, and other rotten organic matter. In addition to eating rotten organic matter, the black soldier fly larvae are used as feed for chickens and pigs as a source of protein.

Nowadays, people breed black soldier flies artificially to increase the production of black soldier fly larvae, which can be used to process waste effectively and convert it into protein for animal feed, thereby achieving the purpose of “sustainable agriculture”. When breeding black soldier flies, people need to pay attention to the humidity and temperature of the breeding space or the number of black soldier flies per unit volume in the breeding space. When the humidity is too low, adult black soldier flies will become dehydrated and the egg-laying rate will decrease. When the temperature is too high, the black soldier fly larvae die easily.

In the process of breeding black soldier fly larvae, the larvae will eat the feed or leave the feed for a long time, which will cause the feed to ferment and raise the temperature of the breeding space. In order to avoid high temperature affecting the growth of larvae, Taiwan Utility Model Publication No. M586048 discloses an improved stirring mechanism of an automated breeding device for black soldier flies. A stirring unit is provided on a movable mechanism installed above a breeding tank. The front end or/and the rear end of the movable mechanism are provided with a smoothing unit and a collecting unit, respectively. When the feed is mixed and stirred, the insect fertilizer and natural waste materials in the breeding tank are cut, stacked and concentrated along with the displacement of the movable mechanism and the continuous stirring of the mixing unit, in cooperation with the lowering of the smoothing unit or collecting unit, such that the stirring unit can stir and mix the insect fertilizer and natural waste materials in the breeding tank to avoid the tamping phenomenon affecting the healthy growth of the black soldier fly.

Taiwan Utility Model Publication No. M622613 discloses a black soldier fly breeding apparatus and a stirring device thereof. The stirring device includes a movable mechanism located above a black soldier fly breeding tank. A stirring mechanism is mounted on the movable mechanism. The stirring mechanism includes a lifting unit, a lifting base mounted on the lifting unit, and a stirring unit mounted on the lifting base. The stirring unit has a plurality of elongate stirring rods extending outwardly from a rotating shaft thereof. When the lifting base moves downward from the raised position to the stirring position, the stirring unit will be driven to move downward and the feed will be stirred by the stirring rods. The stirring rods of the stirring unit are in the form of an elongate structure to reduce the resistance of the stirring rods greatly when stirring the feed. The stirring unit can be driven by a lower-horsepower drive unit, which is beneficial to reduce the size and cost of the apparatus.

However, the above-mentioned stirring mechanisms are too complicated and require more labor and time to perform maintenance at high cost.

### SUMMARY OF THE INVENTION

The primary object of the present invention is to provide an automatic breeding control system for black soldier flies, which simplifies the stirring equipment required for breeding black soldier fly larvae and automatically collects the black soldier fly larvae.

In order to achieve the foregoing object, the present invention provides an automatic breeding control system for black soldier flies, comprising a conveying unit, a lifting unit, at least one infrared temperature sensor and a control unit. The conveying unit has a conveying base and a conveying surface. The conveying surface is movably disposed on the conveying base in a horizontal direction. Two side baffles extending in the horizontal direction are provided on the conveying base. The side baffles are located on two opposite sides of the conveying surface, respectively. The side baffles and the conveying surface together define a breeding space and two outlets located at two opposite ends in the horizontal direction. The outlets communicate with the breeding space, respectively. The lifting unit is selectively moved closer to or away from the conveying unit in a height direction. The lifting unit has two outlet baffles corresponding in position to the outlets. The lifting unit further has a plurality of rakes that are arranged along the horizontal direction between the outlet baffles. The infrared temperature sensor is disposed on the lifting unit and adjacent to the breeding space. The control unit is in signal connection with the conveying unit, the lifting unit and the infrared temperature sensor. The infrared temperature sensor detects a breeding temperature of the breeding space. When the breeding temperature reaches a first temperature, the control unit controls the lifting unit to move closer to the conveying unit, so that the outlet baffles close the respective outlets, and the rakes extend into the breeding space to be selectively adjacent to or in contact with the conveying surface; the control unit controls the conveying surface to move back and forth along the horizontal direction, so that black soldier fly larvae in the breeding space are stirred. When the infrared temperature sensor detects that the breeding temperature of the breeding space drops to a second temperature, the control unit controls the conveying surface to stop moving.

Preferably, the first temperature is 35 degrees Celsius.

Preferably, the second temperature is 30 degrees Celsius.

Preferably, the side baffles each have a bottom portion and an upright portion that are perpendicular to each other. The bottom portion is against the conveying base. The upright portions of the side baffles are arranged facing each other.

Preferably, the number of the rakes is four. The rakes are arranged at equal intervals in sequence along the horizontal direction.

Preferably, the conveying unit further has a receiving plate. The receiving plate is located adjacent to one of the outlets and is lower than the conveying surface in the height direction. The receiving plate has a receiving slope having two opposite ends. One of the two ends of the receiving plate, away from the conveying surface, has an exit.

According to the above technical features, the present invention achieves the following effects:
1. The present invention can detect the temperature of the breeding space, and stir the feed and the black soldier fly larvae in the breeding space timely, so that the feed will not be heated up or caked by moisture. This can prevent the black soldier fly larvae from dying due to the increase in temperature caused by the fermentation of the feed.
2. The slender rakes can reach into the breeding space to stir the feed and the black soldier fly larvae in the breeding space. The rakes have less resistance when they are pushed, which can reduce the load on the conveying unit driving the conveying surface and prevent the load from overheating and causing the temperature of the whole conveying unit to rise and affect the growth of the black soldier fly larvae.
3. When the conveying surface moves back and forth in the horizontal direction, the outlet baffles can close the outlets effectively, so that the black soldier fly larvae will not spill out of the outlets when shaking.
4. When the black soldier fly larvae grows to the final instar stage, the black soldier fly larvae in the breeding space are moved to leave the conveying surface via one of the outlets and fall on the receiving plate to pass through the exit and fall into a container. The black soldier fly larvae that have grown to the final instar stage can be collected easily.

### BRIEF DESCRIPTION OF THE DRAWINGS

FIG. 1 is a perspective view of the present invention;

FIG. 2 is a front view of the present invention;

FIG. 3 is a block diagram of the present invention;

FIG. 4 is a schematic view of the present invention when in use, wherein the lifting unit is moved closer to the conveying unit;

FIG. 5 is a perspective view of FIG. 3;

FIG. 6 is a schematic view of the present invention when in use, wherein the conveying surface of the conveying unit moves back and forth along the horizontal direction; and

FIG. 7 is a schematic view of the present invention when in use, wherein the black soldier fly larvae in the breeding space are moved out.

### DETAILED DESCRIPTION OF THE PREFERRED EMBODIMENTS

Embodiments of the present invention will now be described, by way of example only, with reference to the accompanying drawings.

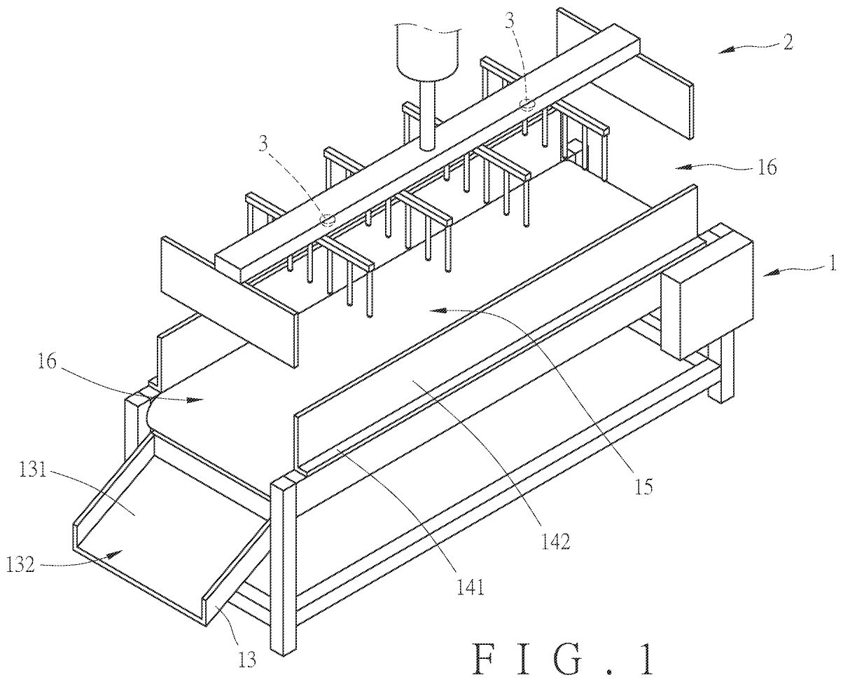

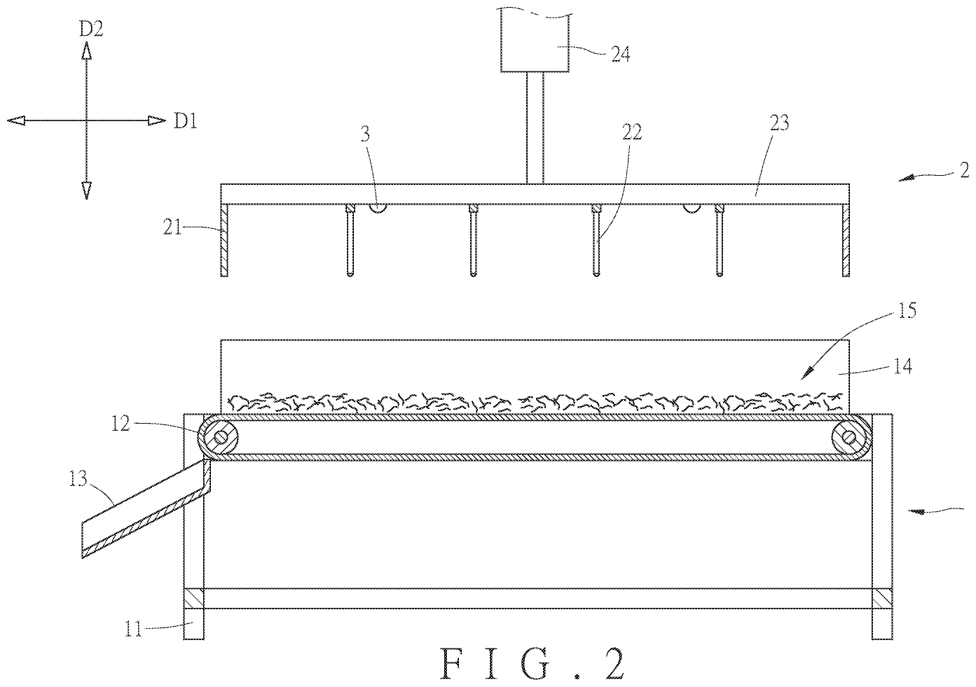

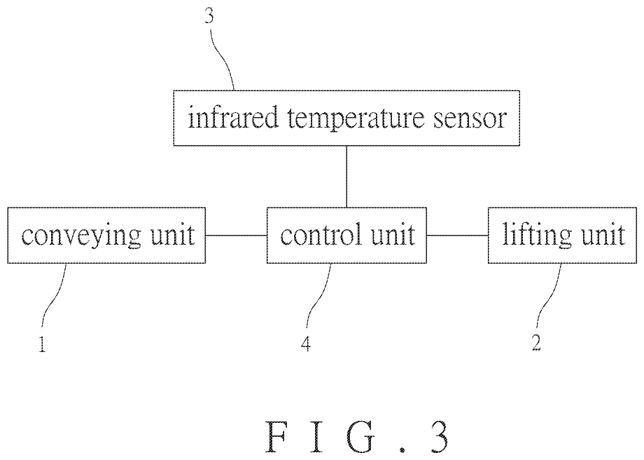

Referring to FIG. 1, FIG. 2 and FIG. 3, the present invention discloses an automatic breeding control system for black soldier flies, comprising a conveying unit 1, a lifting unit 2, two infrared temperature sensors 3, and a control unit 4.

The conveying unit 1 has a conveying base 11, a conveying surface 12, and a receiving plate 13. The conveying surface 12 is movably disposed on the conveying base 11 in a horizontal direction D1. Two side baffles 14 extending in the horizontal direction D1 are provided on the conveying base 11. The side baffles 14 are located on two opposite sides of the conveying surface 12, respectively. The side baffles 14 and the conveying surface 12 together define a breeding space 15 and two outlets 16 located at two opposite ends in the horizontal direction D1. The outlets 16 communicate with the breeding space 15, respectively. The receiving plate 13 is disposed adjacent to one of the outlets 16 and is lower than the conveying surface 12 in a height direction D2. Specifically, the side baffle 14 has a bottom portion 141 and an upright portion 142 that are perpendicular to each other. The bottom portion 141 is against the conveying base 11. The upright portions 142 of the side baffles 14 are arranged facing each other. The conveying surface 12 is disposed between the two upright portions 142. The receiving plate 13 has a receiving slope 131. One end of the receiving plate 13, away from the conveying surface 12, has an exit 132.

The lifting unit 2 is selectively moved closer to or away from the conveying unit 1 in the height direction D2. The lifting unit 2 has two outlet baffles 21 corresponding in position to the outlets 16. The lifting unit 2 further has a plurality of rakes 22 that are arranged along the horizontal direction D1 between the outlet baffles 21. Specifically, the lifting unit 2 has a bracket 23 and a pneumatic cylinder 24. The pneumatic cylinder 24 is connected to the bracket 23. The pneumatic cylinder 24 is fixed above the conveying unit 1 in the height direction D2. The two outlet baffles 21 and the four rakes 22 are fixed to the bracket 23. The two outlet baffles 21 are fixed to two opposite ends of the bracket 23, respectively. The rakes 22 are arranged at equal intervals in sequence along the horizontal direction D1.

The infrared temperature sensor 3 is fixed on the bracket 23 and adjacent to the breeding space 15. Specifically, the infrared temperature sensor 3 is disposed between two of the rakes 22.

The control unit 4 is in signal connection with the conveying unit 1, the lifting unit 2 and the infrared temperature sensor 3.

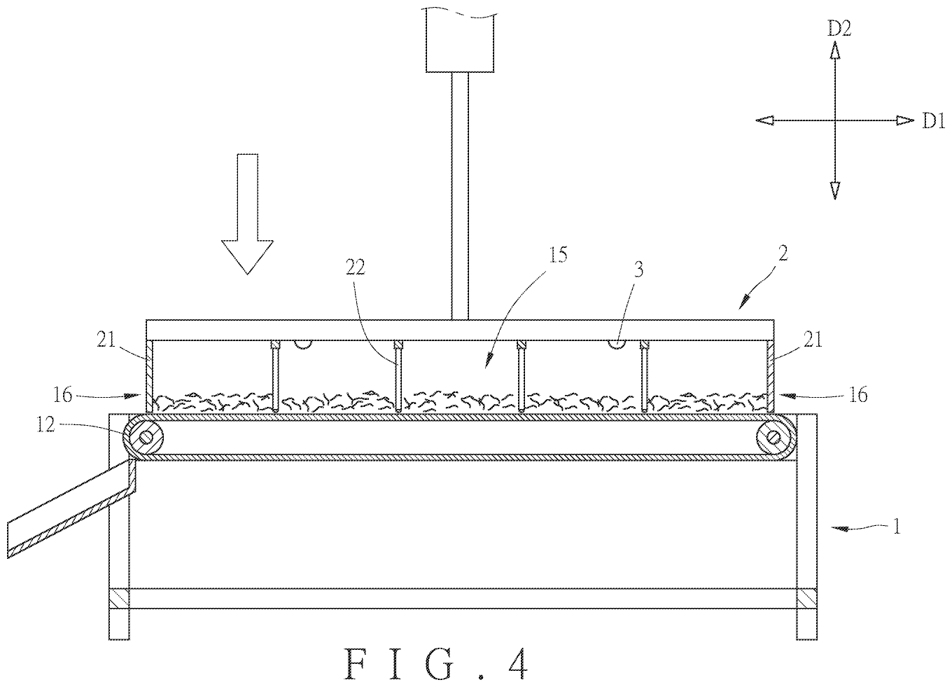

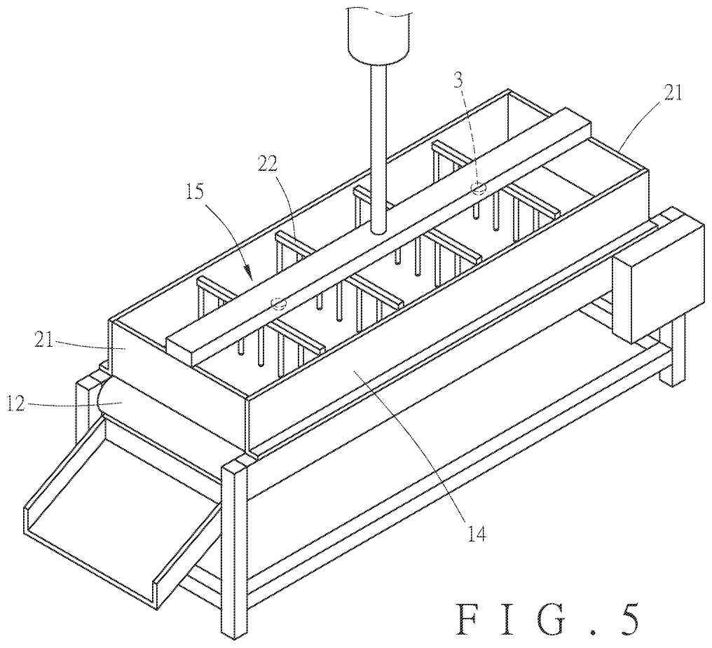

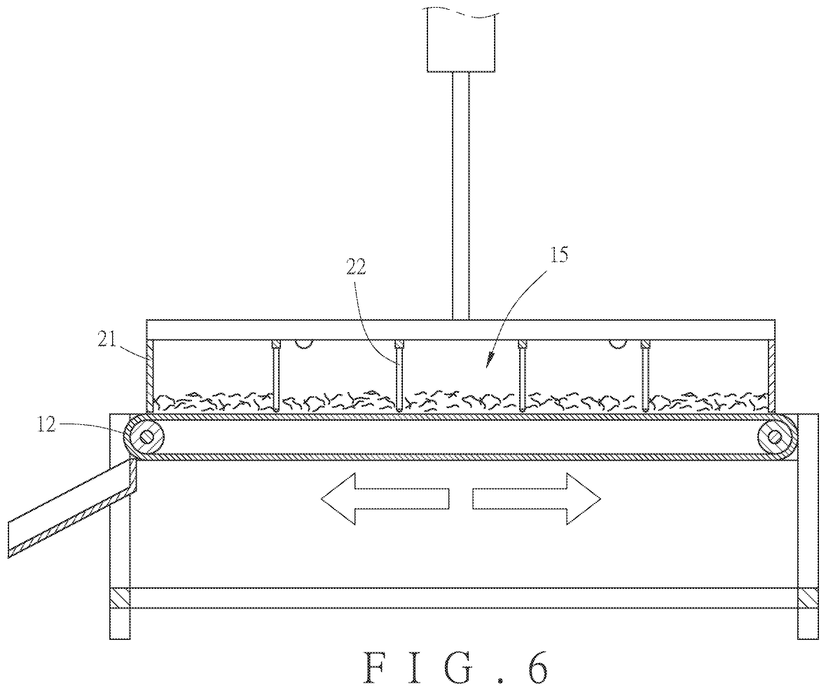

Please refer to FIG. 3, FIG. 4, FIG. 5 and FIG. 6. When the automatic breeding control system of the present invention is in use, the black soldier fly larvae and the feed are placed in the breeding space 15. The feed, such as bean dregs or rice bran powder, is mixed with water to form a viscous consistency. When the black soldier fly larvae and the feed are placed in the same breeding space 15, the feed will ferment to be at high temperature after a long time, or the temperature of the breeding space 15 will increase because the black soldier fly larvae, feed, and excreta are piled on top of each other. Therefore, the infrared temperature sensor 3 is configured to detect a breeding temperature of the breeding space 15. When the breeding temperature reaches a first temperature (in this embodiment, the first temperature is 35 degrees Celsius), the control unit 4 controls the lifting unit 2 to move closer to the conveying unit 1, so that the outlet baffles 21 close the respective outlets 16, and the rakes 22 extend into the breeding space 15 to be selectively adjacent to or in contact with the conveying surface 12. At the same time, the control unit 4 controls the conveying surface 12 to move back and forth along the horizontal direction D1, so that the black soldier fly larvae in the breeding space 15 are stirred. When the infrared temperature sensor 3 detects that the breeding temperature of the breeding space 15 drops to a second temperature (in this embodiment, the second temperature is 30 degrees Celsius), the control unit 4 controls the conveying surface 12 to stop moving and controls the lifting unit 2 to move away from the conveying unit 1. The infrared temperature sensor 3 detects the breeding temperature of the breeding space 15, and the black soldier fly larvae and feed in the breeding space 15 are stirred in a timely manner to prevent the black soldier fly larvae from dying due to high temperature of the breeding space 15.

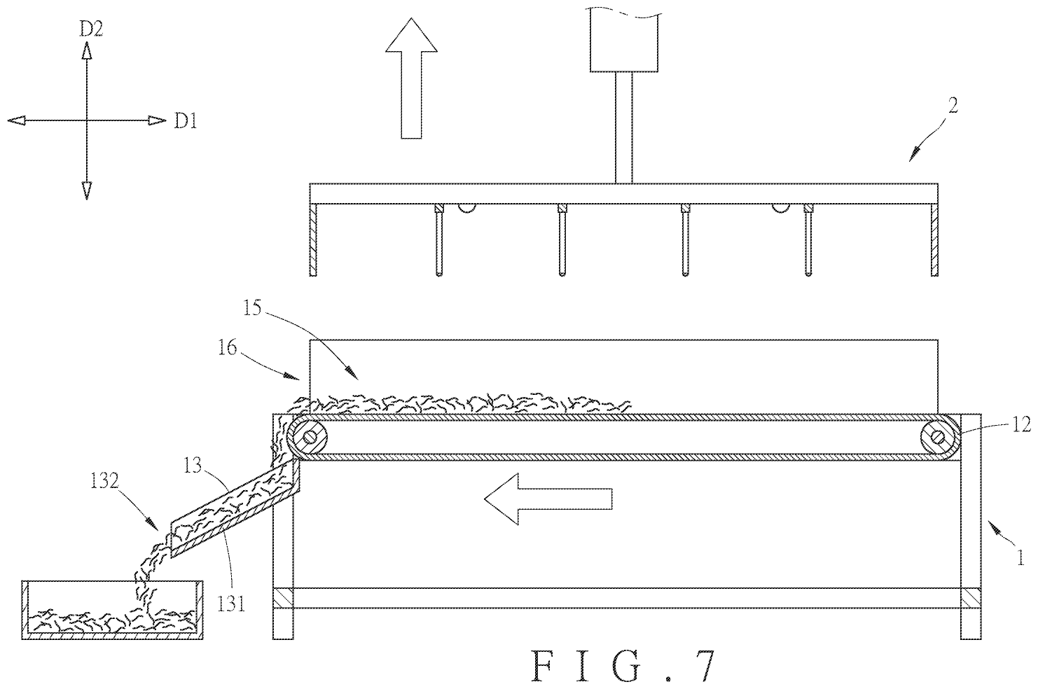

Please refer to FIG. 3 and FIG. 7. When the black soldier fly larvae grows to the final instar stage, the control unit 4 controls the lifting unit 2 to move away from the conveying unit 1 and controls the conveying surface 12 of the conveying unit 1 to run continuously, such that the black soldier fly larvae in the breeding space 15 are moved to leave the conveying surface 12 via one of the outlets 16 and fall on the receiving slope 131 of the receiving plate 13 to pass through the exit 132 and fall into a container. By controlling the continuous running of the conveying surface 12 of the conveying unit 1, the black soldier fly larvae that have grown to the final instar stage can be collected easily.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. An automatic breeding control system for black soldier flies, comprising:
a conveying unit, having a conveying base and a conveying surface, the conveying surface being movably disposed on the conveying base in a horizontal direction; two side baffles extending in the horizontal direction being provided on the conveying base, the side baffles being located on two opposite sides of the conveying surface respectively, the side baffles and the conveying surface together defining a breeding space and two outlets located at two opposite ends in the horizontal direction, the outlets communicating with the breeding space, respectively;
a lifting unit, being selectively moved closer to or away from the conveying unit in a height direction, the lifting unit having two outlet baffles corresponding in position to the outlets, the lifting unit further having a plurality of rakes that are arranged along the horizontal direction between the outlet baffles;
at least one infrared temperature sensor, disposed on the lifting unit and adjacent to the breeding space;
a control unit, being in signal connection with the conveying unit, the lifting unit and the infrared temperature sensor;
wherein the infrared temperature sensor detects a breeding temperature of the breeding space, when the breeding temperature reaches a first temperature, the control unit controls the lifting unit to move closer to the conveying unit, so that the outlet baffles close the respective outlets, and the rakes extend into the breeding space to be selectively adjacent to or in contact with the conveying surface; the control unit controls the conveying surface to move back and forth along the horizontal direction, so that black soldier fly larvae in the breeding space are stirred;
wherein when the infrared temperature sensor detects that the breeding temperature of the breeding space drops to a second temperature, the control unit controls the conveying surface to stop moving.

2. The automatic breeding control system as claimed in claim 1, wherein the first temperature is 35 degrees Celsius.

3. The automatic breeding control system as claimed in claim 1, wherein the second temperature is 30 degrees Celsius.

4. The automatic breeding control system as claimed in claim 1, wherein the side baffles each have a bottom portion and an upright portion that are perpendicular to each other, the bottom portion is against the conveying base, and the upright portions of the side baffles are arranged facing each other.

5. The automatic breeding control system as claimed in claim 1, wherein the number of the rakes is four, and the rakes are arranged at equal intervals in sequence along the horizontal direction.

6. The automatic breeding control system as claimed in claim 1, wherein the conveying unit further has a receiving plate, the receiving plate is located adjacent to one of the outlets and is lower than the conveying surface in the height direction, the receiving plate has a receiving slope having two opposite ends, one of the two ends of the receiving plate, away from the conveying surface, has an exit.
